(19) United States
(12) Patent Application Publication
(10) Pub. No.: US 2010/0273756 A1
(43) Pub. Date: Oct. 28, 2010

(54) ADJUNCTIVE FORMULATION AND METHODS FOR PALLIATION OF FINE WRINKLES, MOTTLED HYPERPIGMENTATION, TACTILE ROUGHNESS OF FACIAL SKIN AND RELATED DISORDERS

(76) Inventor: Reza Babapour, Beverly Hills, CA (US)

Correspondence Address: KAREN S. CANADY;CANADY & LORTZ LLP, 4201 Wilshire BLV Suite 622, LOS ANGELES, CA, 90010, US

(21) Appl. No.: 12/766,793

(22) Filed: Apr. 23, 2010

Related U.S. Application Data
(60) Provisional application No. 61/214,640, filed on Apr. 27, 2009.

(51) Int. Cl. A61K 31/56; A61K 31/20; A61P 17/00

(52) U.S. Cl. 514/171; 514/559

(57) ABSTRACT

The present invention is generally directed to the treatment of fine wrinkles, hyperpigmentation, roughness of facial skin, improvement of photoaging, repair of sun damaged skin, and related disorders. In a formulation aspect, the present invention provides a formulation for the adjunct treatment of skin disorders, where the formulation includes tretinoin and azelaic acid. In a method aspect, the present invention provides a method for treating skin disorders. The skin disorders include fine wrinkles, freckles, solar lentigo, skin photodamage, aging process as reflected by changes in a patient's skin, Acne Vulgaris and Melasma or Post-inflammatory hyperpigmentation. The method includes the application of a formulation to a portion of the patient's skin, where the portion exhibits a skin disorder, and wherein the formulation includes tretinoin and azelaic acid.

This application claims the benefit of U.S. provisional patent application No. 61/214,640, filed Apr. 27, 2009, the entire contents of which are incorporated herein by reference.

### FIELD OF THE INVENTION

The present invention is generally directed to the treatment of fine wrinkles, freckles, solar lentigo, skin photodamage, the aging process as reflected by changes in a patient's skin, Acne Vulgaris, Melasma and Post-inflammatory hyperpigmentation.

### BACKGROUND OF THE INVENTION

RENOVA™ (tretinoin cream) 0.05% is an adjunctive agent for use in the mitigation (palliation) of fine wrinkles, mottled hyperpigmentation and tactile roughness of facial skin in patients who use comprehensive skin care and sunlight avoidance programs. RENOVA™ does not eliminate wrinkles, repair sun damaged skin, reverse photoaging, or restore more youthful or younger skin. In double-blinded, vehicle-controlled clinical studies, many patients in the vehicle group achieved palliative effects on fine wrinkling, mottled hyperpigmentation, and tactile roughness of facial skin with the use of comprehensive skin care and sunlight avoidance programs including sunscreens, protective clothing, and non-prescription emollient creams.

RENOVA 0.05% has not demonstrated a mitigating effect on deep wrinkling, skin yellowing, lentigines, telangiectasia, skin laxity, keratinocyte atypia, melanocytic atypia or dermal elastosis, which are oftentimes the result of chronic sun exposure. The effectiveness of RENOVA 0.05% for mitigating fine wrinkles, mottled hyperpigmentation and tactile roughness of facial skin has not been established in people greater than 50 years of age.

There is a need for improved compounds, formulations and methods that can address the same indications as RENOVA.

### SUMMARY OF THE INVENTION

The present invention is generally directed to the treatment of fine wrinkles, hyperpigmentation, roughness of facial skin, improvement of photoaging, repair of sun damaged skin, and related disorders.

In a formulation aspect, the present invention provides a formulation for the adjunct treatment of skin disorders, where the formulation includes tretinoin and azelaic acid.

In a method aspect, the present invention provides a method for treating skin disorders. The skin disorders include fine wrinkles, freckles, solar lentigo, skin photodamage, aging process as reflected by changes in a patient's skin, Acne Vulgaris and Melasma or Post-inflammatory hyperpigmentation. The method includes the application of a formulation to a portion of the patient's skin, where the portion exhibits a skin disorder, and wherein the formulation includes tretinoin and azelaic acid.

### DETAILED DESCRIPTION OF THE INVENTION

The present invention is generally directed to the treatment of fine wrinkles, hyperpigmentation, roughness of facial skin, improvement of photoaging, repair of sun damaged skin, and related disorders. It is more specifically directed to formulations that include both tretinoin and azelaic acid, which are used to treat a patient's skin.

Formulations according to the present invention are typically in one of the following forms: solution, gel (e.g., water-based), cream, emollient, foam, ointment, lotion, shampoo, or spray. In one embodiment the formulation is a fragrance free, alcohol free, water-based gel.

Tretinoin is included in the formulations at a concentration of at least 0.005%. Oftentimes it is included in the formulations at a concentration of at least 0.01%, 0.015%, 0.02%, 0.025%, 0.03%, 0.035%, 0.040%, 0.045%, 0.050%, 0.055%, 0.060%, 0.065%, 0.070%, 0.075%, 0.080%, 0.085%, 0.090%, 0.095%, or 0.10%. Tretinoin concentration in the formulations typically does not exceed 0.5%, and it usually does not exceed 0.45%, 0.40%, 0.35%, 0.30%, 0.25%, 0.20%, 0.15%, 0.10%, or 0.05%.

Azelaic acid is included in the formulations at a concentration of at least 5%. It is oftentimes included in the formulations at a concentration of at least 7.5%, 10.0%, 12.5%, 15.0%, 17.5%, 20.0%, 22.5%, 25.0%, 27.5%, or 30.0%. Azelaic acid concentration in the formulations typically does not exceed 50.0%, and it usually does not exceed 47.5%, 45.0%, 42.5%, 40.0%, 37.5%, 35.0%, 32.50%, 30.0%, 27.50%, 25.0%, 22.5%, or 20%.

Preferred formulations according to the present invention include about 0.02% tretinoin and about 15% azelaic acid or about 0.05% tretinoin and 15% azelaic acid.

The formulations may optionally include one or both of the following: fluocinolone acetonide (0.005%, 0.0075%, 0.01%, 0.015% or 0.02%); hydroquinone (0.5%, 1.0%, 1.5%, 2.0%, 2.5%, 3.0%, 3.5%, 4.0%, 4.5%, 5.0%, 5.5% or 6.0%).

Formulations of the present invention may be used to treat at least the following conditions: fine wrinkles, freckles, solar lentigo, skin photodamage, aging process as reflected by changes in a patient's skin, Acne Vulgaris and Melasma and Post-inflammatory hyperpigmentation.

The formulation is typically applied once daily, although application of the formulation more than once daily may be advisable for certain indications.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A formulation for the adjunct treatment of skin disorders, wherein the formulation comprises tretinoin and azelaic acid.

2. The formulation according to claim 1, wherein the concentration of tretinoin in the formulation is at least 0.01%.

3. The formulation according to claim 2, wherein the concentration of azelaic acid in the formulation is at least 7.5%.

4. The formulation according to claim 3, wherein the concentration of tretinoin in the formulation is at least 0.02% and the concentration of azelaic acid in the formulation is at least 10.0%.

5. The formulation according to claim 4, wherein the concentration of tretinoin in the formulation does not exceed 10%.

6. The formulation according to claim 5, wherein the concentration of azelaic acid in the formulation does not exceed 30%.

7. The formulation according to claim 6, wherein the formulation is a solution, gel, cream, emollient, foam, ointment, lotion, shampoo or spray.

8. The formulation according to claim 7, wherein the formulation is a fragrance free, alcohol free, water-based gel.

9. The formulation according to claim 8, wherein the formulation further comprises fluocinolone acetonide.

10. A method for treating skin disorders, wherein the skin disorders include fine wrinkles, freckles, solar lentigo, skin photodamage, aging process as reflected by changes in a patient's skin, Acne Vulgaris and Melasma or Post-inflammatory hyperpigmentation, and wherein the method comprises the application of a formulation to a portion of the patient's skin, and wherein the portion exhibits a skin disorder, and wherein the formulation comprises tretinoin and azelaic acid.
